(19) United States
(12) Patent Application Publication
(10) Pub. No.: US 2022/0230763 A1
(43) Pub. Date: Jul. 21, 2022

(54) METHOD AND SYSTEM FOR PROVIDING INTERACTIVE MEDICAL GUIDELINE

(71) Applicant: Roche Molecular Systems, Inc., Pleasanton, CA (US)

(72) Inventors: Qianwei XIE, Pleasanton, CA (US); Marc MAILMAN, Pleasanton, CA (US); Vishakha SHARMA, Pleasanton, CA (US); Veronica NEMMER, Pleasanton, CA (US); Elizabeth LITTLE, San Jose, CA (US); Wojciech KACZMAREK, Pleasanton, CA (US); Maciej CHMIELARSKI, Pleasanton, CA (US); Tomasz SCIURKA, Pleasanton, CA (US); Szymon DOLATA, Pleasanton, CA (US); Maciej LASKOWSKI, Pleasanton, CA (US)

(21) Appl. No.: 17/712,928

(22) Filed: Apr. 4, 2022

Related U.S. Application Data
(63) Continuation of application No. PCT/US2020/055483, filed on Oct. 14, 2020.
(60) Provisional application No. 62/914,942, filed on Oct. 14, 2019.

(51) Int. Cl. G16H 70/20; G16H 50/20; G06N 5/00

(52) U.S. Cl. CPC G16H 70/20; G06N 5/003; G16H 50/20

(57) ABSTRACT

Methods and systems for providing an interactive guideline are provided. In one example, a method comprises: receiving, from a database, data of a directed graph representing a medical guideline, the medical guideline including a decision tree including a plurality of clinical decisions and preconditions leading to at least some of the clinical decisions, the directed graph including a plurality of nodes representing the clinical decisions and the preconditions and edges connecting the plurality of nodes to represent dependency relationships among the clinical decisions and the preconditions; providing, via a navigation interface, a graphical representation of at least part of the directed graph; receiving, via the navigation interface, a selection of a node of the directed graph from the graphical representation; and based on the selection of the node, updating the graphical representation of the at least part of the directed graph to provide a navigation result of the medical guideline.

### CROSS-REFERENCES TO RELATED APPLICATIONS

The present application is a continuation of International Application No. PCT/US2020/055483 filed Oct. 14, 2020, which claims benefit of priority to U.S. Provisional Patent Application No. 62/914,942, filed Oct. 14, 2019, the contents of each of which are herein incorporated by reference in their entireties for all purposes.

### BACKGROUND

A medical guideline generally refers to a document with the aim of guiding decisions and criteria regarding diagnosis, management, and treatment in specific areas of healthcare. The medical guideline may provide the most current data about prevention, diagnosis, prognosis, therapy including dosage of medications, risk/benefit and cost-effectiveness of a treatment for a particular disease. The guideline may also identify all available (or known) decision/treatment options and their outcomes at a particular stage of the disease or the treatment, and different options/outcomes at each can be identified at different stages of the disease/treatment. Based on a current medical condition of a patient, a clinician can refer to the guideline to obtain the different treatment options and possible outcomes, and can determine a treatment option for the patient.

Given that a medical guideline identifies all available decision/treatment options and their outcomes at different stages of the disease/treatment, the medical guideline is typically a huge document having hundreds, if not thousands, of pages. For example, the National Comprehensive Cancer Network® (NCCN) Clinical Practical Guidelines in Oncology for Breast Cancer has more than 200 pages. It can be difficult for a clinician to navigate through the huge document and identify the different treatment options/outcomes for a given medical condition. The difficulty is further exacerbated when the clinician needs to navigate through the medical guideline to search for different treatment options for different patients at different stages of the disease/treatment.

### BRIEF SUMMARY

Disclosed herein are techniques for providing an interactive medical guideline engine. The interactive medical guideline engine includes a navigation interface to provide a graphical representation of a medical guideline to a user, such as a medical care provider (e.g., a clinician, a clinician staff member, etc.). The medical guideline may include a decision tree, which includes a set of clinical decisions, such as a treatment, a diagnosis, etc., as well as one or more pre-conditions that leads to a clinical decision within the decision tree. The navigation interface can provide different forms of graphical representation of the medical guideline in different operation modes, such as a tree mode and a step mode. To provide an interactive experience, the navigation interface can also provide different ways of navigating through the medical guideline and selecting clinical decisions in the medical guideline under the different operation modes. The interactive medical guideline engine can also allow the user to store a sequence of clinical decisions selected for a patient, and to add customized treatment/diagnostic tests procedures which are not part of the medical guideline to the sequence. The sequence of clinical decisions selected/added for a patient can be presented as part of treatment history of the patient. The interactive medical guideline engine can also detect an update to the medical guideline, and provide notifications via the navigation interface to the user. The notifications can prompt the user to, for example, remove the sequence of clinical decisions selected for a patient from the old medical guideline, create a new sequence of clinical decisions selected for the patient from the updated medical guideline, etc.

These and other embodiments of the invention are described in detail below. For example, other embodiments are directed to systems, devices, and computer readable media associated with methods described herein.

A better understanding of the nature and advantages of embodiments of the present invention may be gained with reference to the following detailed description and the accompanying drawings.

### BRIEF DESCRIPTION OF THE DRAWINGS

The detailed description is set forth with reference to the accompanying figures.

FIG. 1A and FIG. 1B illustrate examples of a medical guideline.

FIG. 2 illustrates a system for providing improved access to a medical guideline.

FIG. 3A, FIG. 3B, and FIG. 3C illustrate example data structures used by the system of FIG. 2 to provide improved access to a medical guideline, according to certain aspects of this disclosure.

FIG. 4 illustrates an example operation of the system of FIG. 2, according to certain aspects of this disclosure.

FIG. 5A, FIG. 5B, and FIG. 5C illustrate example operations of the system of FIG. 2, according to certain aspects of this disclosure.

FIG. 6A and FIG. 6B illustrate example operations of the system of FIG. 2, according to certain aspects of this disclosure.

FIG. 7A, FIG. 7B, FIG. 7C, and FIG. 7D illustrate example operations of the system of FIG. 2.

FIG. 8A and FIG. 8B illustrate example operations of the system of FIG. 2, according to certain aspects of this disclosure.

FIG. 9 illustrate a method of providing an interactive medical guideline, according to certain aspects of this disclosure.

FIG. 10 illustrates an example computer system that may be utilized to implement techniques disclosed herein.

### DETAILED DESCRIPTION

A medical guideline is typically a document that includes information about the most current data about prevention, diagnosis, prognosis, therapy including dosage of medications, risk/benefit and cost-effectiveness of a treatment for a particular disease. The guideline may identify all available (or known) decision/treatment options and their outcomes at a particular stage of the disease or the treatment, and different options/outcomes at each can be identified at different stages of the disease/treatment. Based on a current medical condition of a patient, a clinician can refer to the guideline to obtain the different treatment options and possible outcomes, and can determine a treatment option for the patient. Given that a medical guideline identifies all available decision/treatment options and their outcomes at different stages of the disease/treatment, the medical guideline is typically a huge document having hundreds, if not thousands, of pages. It can be difficult for a clinician to navigate through the huge document to identify the different treatment options/outcomes for different patients at different stages of the disease/treatment.

Disclosed herein are techniques for providing an interactive medical guideline engine which can address at least some of the issues described above. The interactive medical guideline engine can receive a directed graph data structure representing a medical guideline. The medical guideline may include a decision tree including a set of clinical decisions, such as a treatment, a diagnosis, etc., as well as one or more pre-conditions that leads to a clinical decision within the decision tree. The medical guideline directed graph has nodes and edges, with each node representing a clinical decision (e.g., a treatment, a diagnosis, etc.) or a condition that leads to a clinical decision (e.g., a treatment response, a diagnosis result, etc.), whereas each edge connects two nodes and represents a dependency relationship (e.g., a sequential relationship, a logical relationship, etc.) between the two nodes. The interactive medical guideline engine can further include a navigation interface to provide a graphical representation of the directed graph to a user, such as a medical care provider (e.g., a clinician, a clinician staff member, etc.). The navigation interface can provide different forms of graphical representation of the directed graph in different operation modes, such as a tree mode and a step mode. To provide an interactive experience, the navigation interface can also provide different ways of navigating through the directed graph and selecting nodes from the directed graph, to allow the user to navigate through the medical guideline and to select different steps in the medical guideline.

Specifically, under the tree mode, the navigation interface can display the directed graph in a visual tree format corresponding to the decision tree of a medical guideline. The visual tree include graphical elements which reflect the nodes and edges of the directed graph. The navigation interface can have various features to improve the user's experience of navigating through the decision tree of the medical guideline when operating in the tree mode. For example, the interactive medical guideline engine can receive different inputs via the navigation interface and configure the display of the decision tree based on the inputs. The inputs may include, for example, a zoom-in command, a zoom-out command, a drag command, etc., to select part of the decision tree, or the entirety of the decision tree, for displaying in the navigation interface. In addition, each node in the directed graph is selectable from the navigation interface. The selection of a node can represent a clinical decision being made, or a pre-condition for a clinical decision (e.g., a particular treatment response is detected, a particular diagnosis result is made) is satisfied. The sequence of selection of the nodes can represent a history of clinical decisions, treatment responses, and diagnosis results of a patient, and can be stored as part of the patient's medical journey. The interactive medical guideline engine can detect which of the nodes have been selected by the user and visually emphasize (e.g., based on color, tone, font size, line width, etc.) the selected nodes and the edges that lead to the selected nodes in the navigation interface relative to the nodes and edges that are not selected, to provide a clear visual representation of the patient's medical journey in view of the medical guideline.

Moreover, the interactive medical guideline engine can also track and record a sequence of user's selection of nodes in the decision tree of the medical guideline. Such arrangements can serve different purposes. For example, the recorded sequence, which may represent the patient's medical journey in view of the medical guideline, can be part of the history of medical treatment of that patient. As such, the recorded sequence can be stored in an document (e.g., a patient's report, a meeting presentation, etc.) that forms part of the patient's medical record. As another example, the interactive medical guideline engine can also detect whether a user skips a node representing a treatment step or a diagnosis step required by the medical guideline based on the tracked sequence. Upon detecting the user skipping a node, the interactive medical guideline engine may determine that a required node has been skipped and may perform a pre-determined action. The pre-determined action may include, for example, displaying a warning, not accepting the user's node selection (e.g., not highlighting the selected node) in the navigation interface, etc.

Besides the tree mode, the navigation interface can also operate in a step mode. In the step mode, the navigation interface can display the content (e.g., a clinical decision, a pre-condition, etc.) of a currently-selected node of the directed graph, as well as the content of all the direct child nodes of the currently-selected node. In the step mode, the nodes can be represented as boxes, and the edges between the nodes are not displayed. The direct child nodes can represent, for example, all possible options of the next clinical decision, all possible treatment responses/diagnosis results, etc. The step mode allows the user to traverse the directed graph one node at a time. As in the tree mode, the interactive medical guideline engine can also track and record the sequence of user's selection of the nodes in the step mode. In some examples, the interactive medical guideline engine can cause the navigation interface to display, in a separate panel, a sequence of selection of the nodes by the user, which can provide more guidance or context to the user in selecting the next node.

In addition, the interactive medical guideline engine may include other features to further facilitate the user's access of the medical guideline. For example, the interactive medical guideline engine allows the user to create a customized version of the medical guideline, in which the user can add a new clinical procedure (e.g., a treatment procedure, a diagnosis procedure, etc.) to the medical guideline for a patient. The interactive medical guideline engine can then display the customized medical guideline via the navigation interface. The newly-added clinical procedure can also be recorded into part of the sequence of selected nodes/steps to update the medical record of the patient. In addition, the interactive medical guideline engine can also detect an update to the medical guideline and can provide notifications via the navigation interface to the user. The notifications can prompt the user to, for example, remove the sequence of nodes selected from the old medical guideline for a patient, navigate through the new medical guideline to create a new sequence of nodes for the patients, etc., to ensure that the clinician makes the clinical decisions based on the updated medical guideline.

With the disclosed embodiments, a navigation interface allows a user to access different parts of the medical guideline based on simple commands (e.g., clicking, zoom in, zoom out, drag, etc.), which can substantially simplify the user's access to the medical guideline. Moreover, by tracking and displaying the sequence of user's selection of nodes in the decision tree of the medical guideline, the user (e.g., a clinician) can keep track of the medical journeys for different patients in view of the medical guideline, which allows the clinician to consult the relevant portions of the medical guideline for different patients to determine their treatments. Compared with a case where the clinician has to navigate through the huge medical guideline document to search for relevant portions of the medical guideline for different patients, and separately record the guideline steps undertaken by the patients, the disclosed embodiments provide a unified interface to navigate and search the medical guideline and to record the selected steps for different patients. All these can improve the ease of accessing the relevant medical treatment information from the medical guideline for different patients, which in turn can improve the quality of medical treatments provided to those patients.

### I. An Example Medical Guideline Document

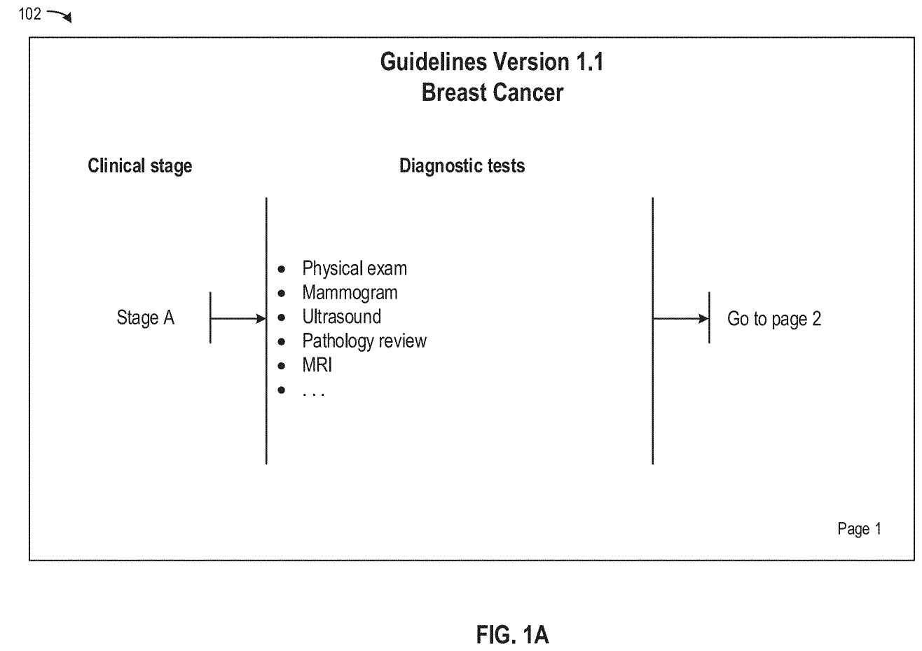

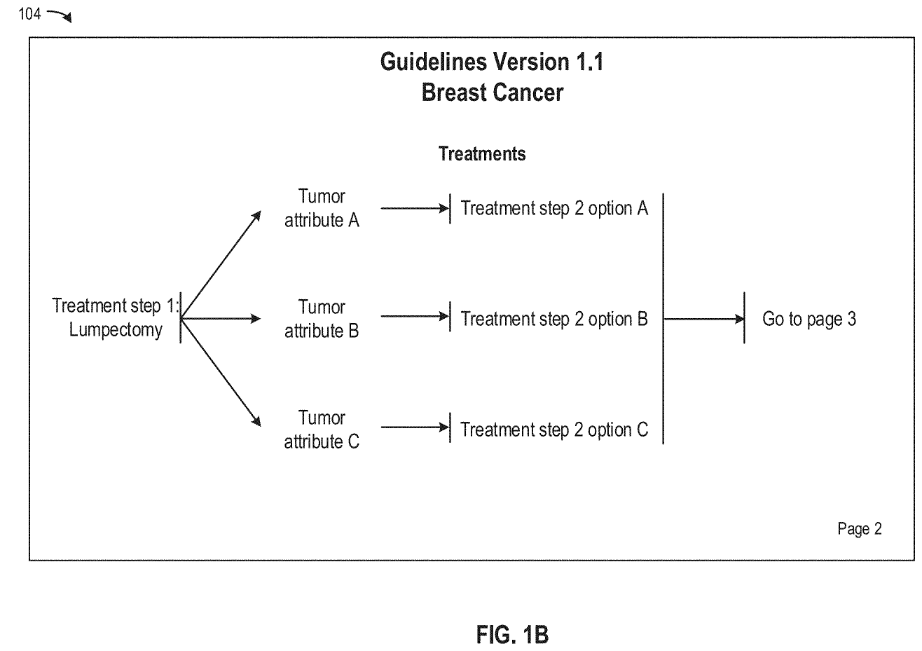

FIG. 1A and FIG. 1B illustrate example excerpts of a conventional medical guideline document. FIG. 1A illustrates a first page 102 of the document, whereas FIG. 1B illustrates a second page 104 of the document. First page 102 and second page 104 illustrates part of a medical guideline for treatment of breast cancer. The medical guideline may include a decision tree, which includes a set of clinical decisions, such as a treatment, a diagnosis, etc., as well as one or more pre-conditions that leads to a clinical decision within the decision tree.

Referring to FIG. 1A, the medical guideline starts with a clinical decision about the patient's clinical stage, and subsequent clinical decisions (e.g., diagnostic tests) following the determination of the patient's clinical stage. First page 102 of the medical guideline states that if the cancer patient is at a clinical stage A, the patient should receive a set of diagnostic tests such as physical examination, mammogram, ultrasound, pathology review, etc., followed by a set of treatments outlined in second page 104.

Referring to FIG. 1B, second page 104 of the medical guideline states that a first treatment step includes a lumpectomy operation to remove the tumor tissues should follow the set of diagnostic tests listed in first page 102. Following the lumpectomy operation, there are three options of the second treatment step. If the cancer tumor has an attribute A, the patient should receive a second treatment step of option A. If the cancer tumor has an attribute B, the patient should receive a second treatment step of option B. If the cancer tumor has an attribute C, the patient should receive a second treatment step of option C. In the example of FIG. 1B, the different attributes of the cancer tumor may refer to, for example, a size of the tumor, whereas the different second treatment step options may include different scopes of radiation therapy. The subsequent treatment/diagnostic tests steps are listed in page 3 of the medical guideline document (not shown in the figures).

Based on a current medical condition of a patient, a clinician can refer to the medical guideline document to obtain the different treatment options and possible outcomes, and can determine a treatment option for the patient. For example, based on the clinical stage of the patient, the clinician may access first page 102 of FIG. 1A to determine the set of diagnostic steps for the patient, followed by second page 104 of FIG. 1B to determine that the patient needs to receive a lumpectomy operation. Moreover, following the lumpectomy operation, depending on the cancer tumor of the patient, the clinician can determine the second treatment step from one of the options listed in second page 104.

While a conventional medical guideline document is an invaluable tool to guide a clinician in prescribing the best (or at least standardized) treatments to the patients, navigating the medical guideline document to obtain the relevant information can be difficult. Specifically, given that a medical guideline identifies all available decision/treatment options and their outcomes at different stages of the disease/treatment, the medical guideline is typically a huge document having hundreds, if not thousands, of pages. For example, the National Comprehensive Cancer Network® (NCCN) Clinical Practical Guidelines in Oncology for Breast Cancer has more than 200 pages. It can be difficult for a clinician to navigate through the huge document and identify the different treatment options/outcomes for a given medical condition. The difficulty is further exacerbated when the clinician needs to navigate through the medical guideline to search for different treatment options for different patients at different stages of the disease/treatment.

Moreover, there is also a need for a convenient and intuitive way to record the treatment/diagnostic tests steps selected by the clinician from the medical guideline document. The recording can be part of the medical treatment history of the patient. Currently, while the clinician can make separate notes to record the treatment/diagnostic tests steps selected from the medical guideline document while reading the document, the record keeping can become unmanageable when the recording is done for a large number of patients.

### II. Interactive Medical Guideline Engine

### A. System Overview

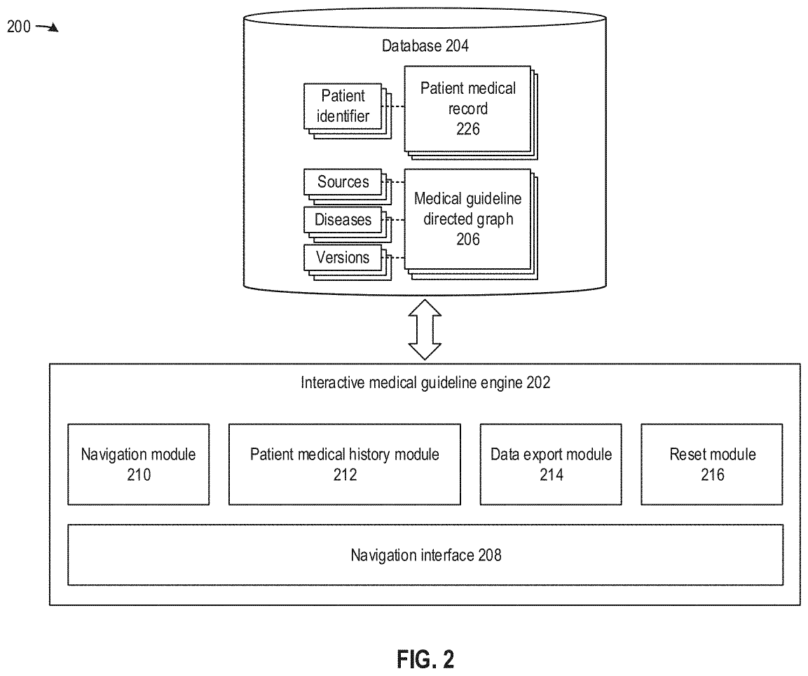

FIG. 2 illustrates a system 200 that can provide improved access to a medical guideline. As shown in FIG. 2, system 200 includes an interactive medical guideline engine 202 and a database 204. Database 204 can store data representing multiple medical guideline directed graphs 206 (herein after, “directed graphs 206”), which can be generated from multiple medical guideline documents. The directed graphs can be associated with different publication sources (e.g., NCCN), diseases (e.g., breast cancer, lung cancer, etc.) and versions. Interactive medical guideline engine 202 can include software instructions, which can be executed by a hardware processor to perform the various functions. In some examples, interactive medical guideline engine 202 can operate on a medical informatics platform and interface with database 204 via the medical informatics platform.

Specifically, interactive medical guideline engine 202 can include a navigation interface 208 to provide a graphical representation of directed graph 206. Navigation interface 208 can provide different forms of graphical representation of directed graph 206 in different operation modes, such as a tree mode and a step mode. Moreover, to provide an interactive experience, navigation interface 208 also provides different ways of navigating through directed graph 206 and making a selection from the graph, to allow the user to navigate through the medical guideline to compile a set of clinical decisions.

As to be described below, interactive medical guideline engine 202 includes navigation module 210, which can detect an input via navigation interface 208. The input may include, for example, a command to navigate to zoom into or zoom out of a particular portion of the medical guideline, to move to a particular portion of the medical guideline, to select a particular treatment/diagnostic test step displayed in navigation interface 208, etc. Based on the input, navigation module 210 can configure the display of the graphical representation of directed graph 206 in navigation interface 208 to provide an interactive experience.

### B. Medical Guideline Directed Graph

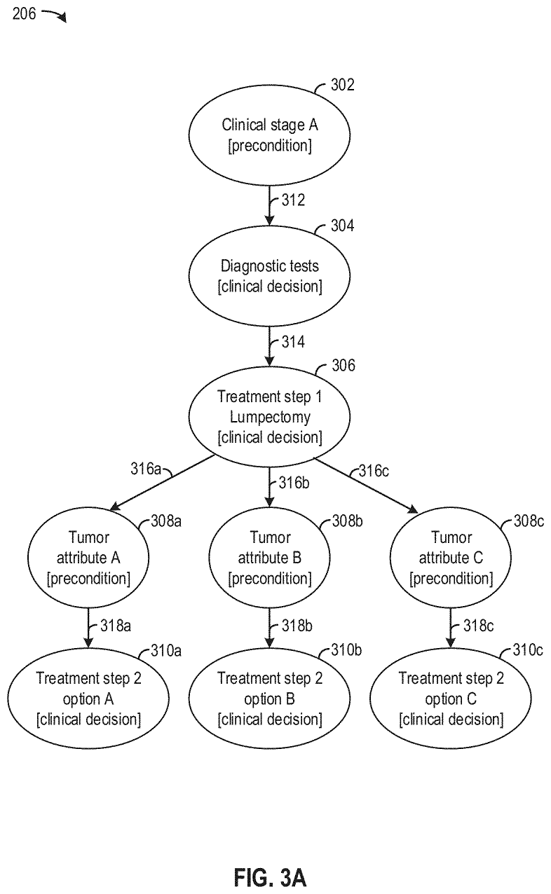

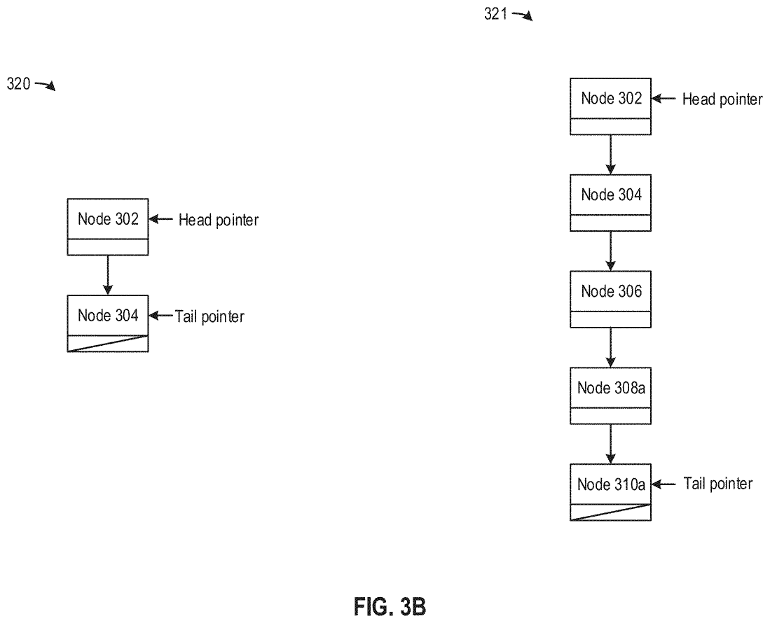

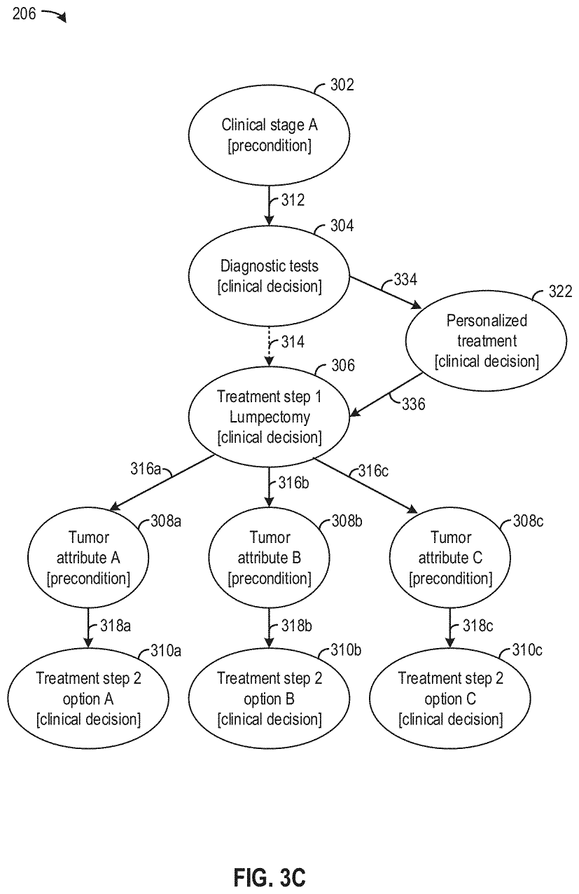

FIG. 3A illustrates an example of a directed graph 206 which can represent part of a medical guideline. The example shown in FIG. 3A can reflect the content of first page 102 and second page 104 of a medical guideline document in FIG. 1A and FIG. 1B. As shown in FIG. 3A, directed graph 206 include multiple nodes including root node 302, child nodes 304, 306, 308a, 308b, 308c, 310a, 310b, and 310c. The nodes are connected by edges, which can represent a dependency relationship among the nodes. Each node can represent a treatment step, a diagnostic test step, a result of the treatment step, a diagnosis result, etc.

In directed graph 206, each node can be associated with a flag (enclosed in square brackets) indicating whether the node is a precondition (e.g., where the node represents a result of the treatment step, a diagnosis result, etc.) or a clinical decision when the precondition is satisfied (e.g., to perform a treatment step, a diagnostic test step, etc.). Each edge can represent a dependency relationship among the nodes, which in turn can reflect, for example, a sequential relationship between two steps, a logical relationship between a precondition and a clinical decision, etc. Directed graph 206 can be generated by extracting the guideline information from a medical guideline document. The extraction can be performed manually, by an automated process, or by a combination of both. Directed graph 206 can also be associated with other information in database 204, such as version information.

Specifically, in the example of FIG. 3A, root node 302 can represent clinical stage A of FIG. 1A, which can be a precondition leading to child node 304, which represents a set of diagnostic tests and is a clinical decision. Root node 302 is connected to child node 304 via edge 312. Child node 304 represents the set of diagnoses that follows clinical stage A in FIG. 1A, while the direction of edge 312 indicates that the set of diagnoses represented by child node 304 follows sequentially from root node 302. Child node 304 is connected to child node 306 via edge 314. Child node 306 represents the first treatment step of lumpectomy of FIG. 1B, another clinical decision. Child node 306 is connected to child nodes 308a, 308b, and 308c via, respectively, edges 316a, 316b, and 316c. Each of child nodes 308a, 308b, and 308c can represent a diagnosis result such as an attribute of the tumor, whereas edges 316a, 316b, and 316c indicate that the checking of the tumor attribute and the treatment steps that follow are to be performed after the lumpectomy operation represented by node 306. Child nodes 308a, 308b, and 308c each is a precondition for an option of a second treatment, represented by child nodes 310a, 310b, and 310c. Specifically, child node 308a is connected to child node 310a, which represents a second treatment of option A (a clinical decision), via edge 318a. Child node 308b is connected to child node 310b, which represents a second treatment of option B (a clinical decision), via edge 318b. Moreover, child node 308c is further connected to child node 310c (a clinical decision), which represents a second treatment of option C, via edge 318c. Each of edges 318a, 318b, and 318c can represent a logical relationship between, respectively, nodes 308a and 310a, nodes 308b and 310b, and nodes 308c and 310c.

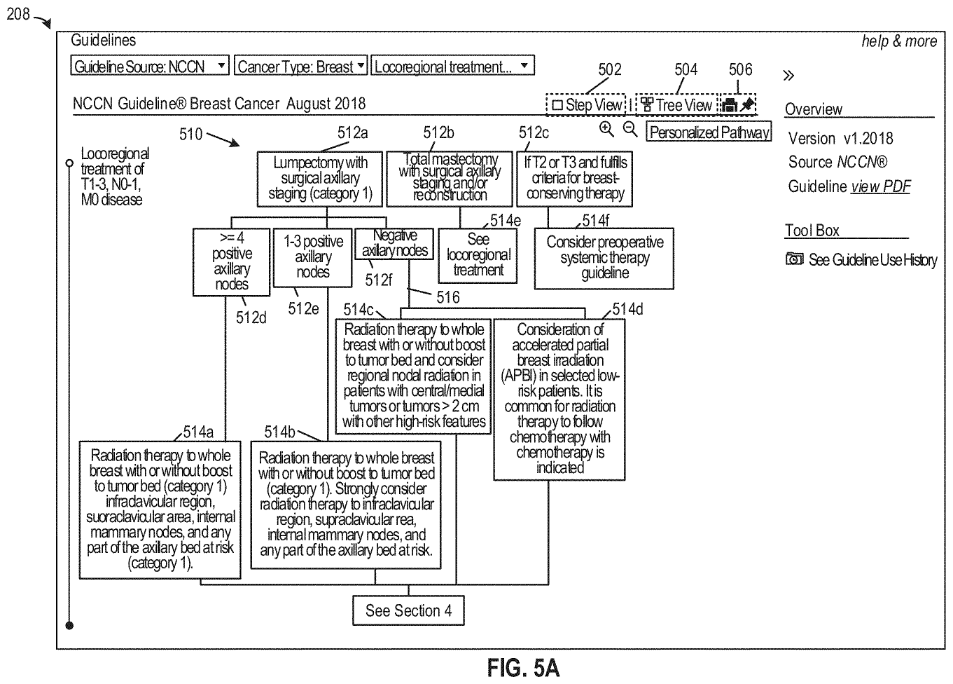

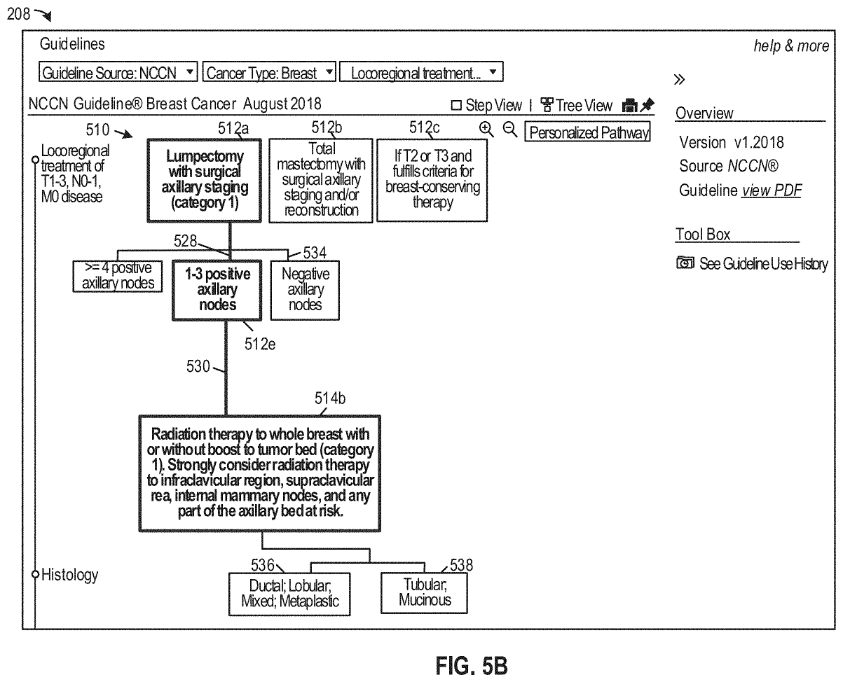

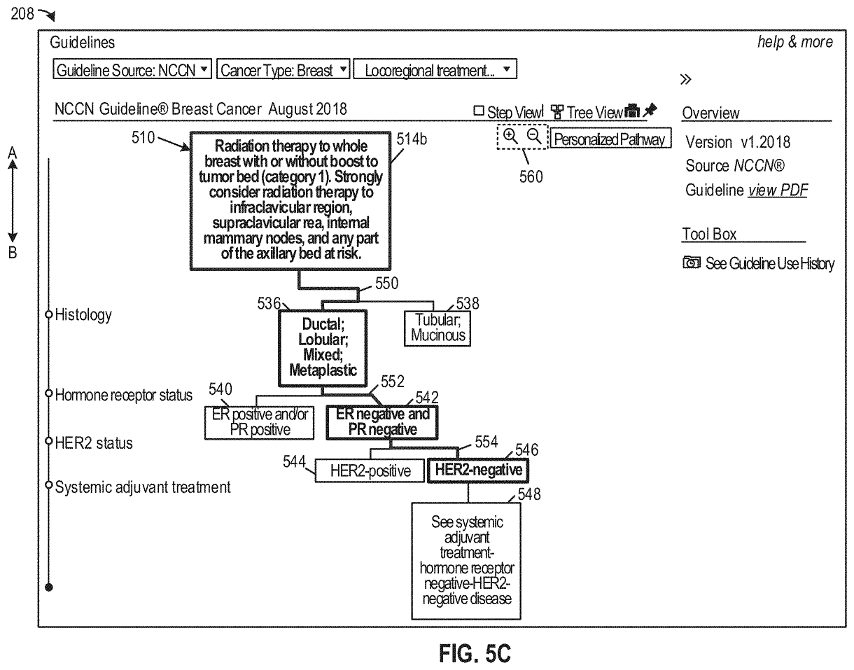

Referring back to FIG. 2, navigation module 210 can retrieve directed graph 206 and provide a graphical representation of the directed graph via navigation interface 208. Navigation module 210 can provide different forms of graphical representation of the medical guideline in different operation modes, such as a tree mode and a step mode. As to be described below, FIG. 5A, FIG. 5B, and FIG. 5C illustrate examples of navigation interface 208 operating under the tree mode. Under the tree mode, the navigation interface can display the directed graph in a decision tree that includes the nodes and edges of directed graph 206. Each node displayed in navigation interface 208 can include the text of, for example, a treatment step, a diagnosis step, a treatment result, a diagnosis result, etc., extracted from the medical guideline document.

Navigation module 210 can also maintain a mapping between each node of the decision tree and different locations within a display frame (e.g., within a frame buffer). The mapping allows a user to control navigation interface 208 to zoom into or out of a node or a set of nodes of the decision tree, to move to a different part of the decision tree that is not currently displayed in navigation interface 208, and/or to select a node as a clinical decision. Specifically, navigation interface 208 can display part of, or the entirety of, the display frame by default.

Navigation interface 208 can receive an input to select a region of the display frame to be displayed by navigation interface 208. Based on the mapping between the nodes and different regions of the display frame, as well as the selected region according to the input, navigation module 210 can identify the nodes and edges of directed graph 206 being selected by the input for displaying. Navigation module 210 can provide the identified nodes and edges to navigation interface 208 for displaying. The input may include, for example, a selection of a node to start the navigation, a zoom-in command to zoom into a particular region of the display frame, a zoom-out command to zoom out of a currently-displayed location of the display frame, a drag command to move to a different regions of the display frame, etc., to change the nodes and edges to be displayed in navigation interface 208.

In addition, to further enhance the user's experience in navigating through the medical guideline, each node in the decision tree displayed in navigation interface 208 is made selectable by interactive medical guideline engine 202. The selection can reflect, for example, a clinical decision (e.g., a treatment or a diagnosis step), a pre-condition to a clinical decision (e.g., a treatment response, a diagnosis result, etc.) being satisfied, etc. Navigation module 210 can detect an input indicating a selection of a node (e.g., a click action) at a particular location of the display frame of navigation interface 208. Based on the mapping between the nodes and the different regions of the display frame, navigation module 210 can identify the node being selected. Based on identifying the node being selected, navigation module 210 can update the display of the decision tree based on the selection. For example, as shown in FIG. 5B and FIG. 5C, navigation module 210 can cause navigation interface 208 to visually emphasize (e.g., based on color, tone, font size, line width, etc.) the selected nodes and the edges that lead to the selected nodes in navigation interface 208 relative to the nodes and edges that are not selected.

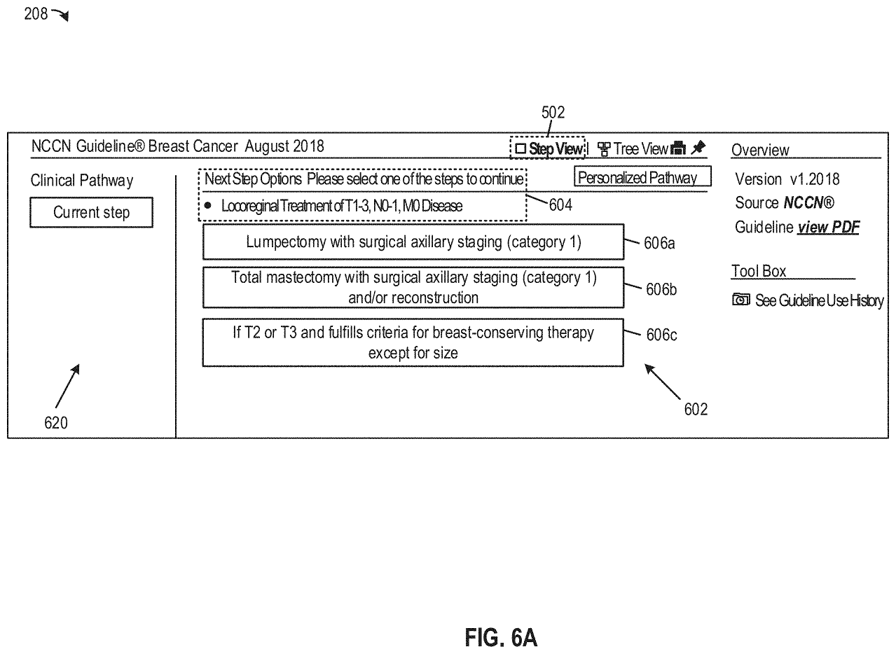

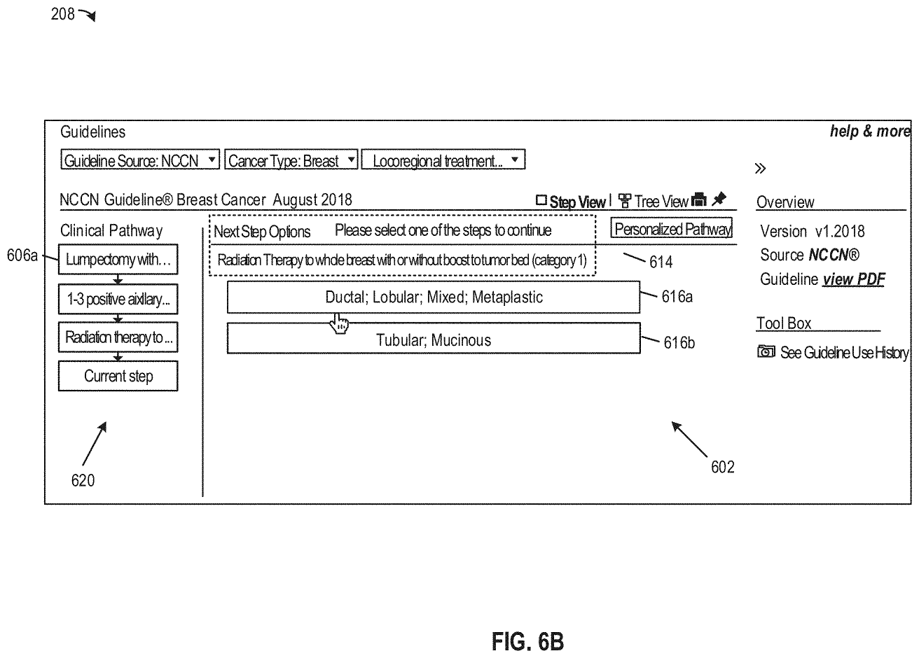

Besides the tree mode, navigation interface 208 can also operate in a step mode. In the step mode, navigation module 210 can cause navigation interface 208 to display a currently-selected node and the direct child nodes of the currently-selected node. In the step mode the nodes can be represented as boxes, and the edges between the nodes are not displayed. The direct child nodes can represent, for example, all possible options of the next clinical decision, all possible treatment responses/diagnosis results, etc. The user can navigate the medical guideline in the step mode by selecting one node from the direct child nodes at a time. Based on the selected node and the location of the selected node in the directed graph, navigation module 210 can change the display of the currently-selected node and the direct child nodes in navigation interface 208. As to be described below, FIG. 6A and FIG. 6B illustrate example operations of navigation interface 208 under the step mode.

### C. Selection Sequence Recording

Moreover, navigation module 210 can track and record a sequence of user's selection of nodes of the medical guideline, in both the tree mode and the step mode. The sequence can be stored in a linear data structure, such as a linked list. FIG. 3B illustrates example linked lists 320 and 321, which can record a sequence of user's selection of nodes in the decision tree maintained by navigation module 210. As shown in FIG. 3B, each of linked lists 320 and 321 includes multiple elements, with each element corresponding to a selected node. Each element can store (or links to) text describing a precondition (e.g., a treatment response, a diagnostic result) or a clinical decision (e.g., a treatment step, a diagnostic test, etc.). Each element can also store (or links to) a timestamp which indicate a date of the selection of the node. Each linked list has a head pointer and a tail pointer. The head pointer points to a first element of the linked list which represents the first node selected by the user, whereas the tail pointer points to the latest element added to the linked list which represents the last node selected by the user. Each element is added to the linked list based on the order by which the corresponding node is selected by the user. Navigation module 210 can store the linked lists to represent different sequences of treatment and diagnosis steps as well as treatment and diagnosis results selected from the medical guideline. For example, as shown in FIG. 3B, linked list 320 indicates that the user has made a sequence of selections, from navigation interface 208, of node 302 followed by node 304 of directed graph 206. Moreover, linked list 321 indicates that the user has made a sequence of selections, from navigation interface 208, of nodes 302, 304, 306, 308a, and 310a of directed graph 206.

The recording of sequences of selection of the nodes can serve various purposes. In one example, the recording of a sequence of selection can be used by navigation module 210 to validate the user's selection of a node from directed graph 206. Specifically, the selection of the nodes (and the corresponding treatment/diagnostic tests steps) from the directed graph 206 need to follow the sequential or logical relationships defined in the medical guideline. For example, referring to directed graph 206 of FIG. 3A, after the user selects node 306 representing the first treatment step of lumpectomy, the user should then select one of nodes 308a, 308b, or 308c (tumor attributes A, B, or C). The user should not skip those nodes and subsequently select, for example, any one of nodes 310a, 310b, or 310c. Moreover, after the user selects node 308a (tumor attribute A), the user should select node 310a (second treatment step of option A), not node 310b or 310c (second treatment step options B or C).

When receiving a selection of a new node from a user, navigation module 210 can validate the selection by retrieving the last selected node from the linked list (the node corresponding to the last element of the linked list) and determining whether the new node immediately follows the last selected node in guideline directed graph 206 (e.g., whether the two nodes are connected by one edge or separated by other nodes). If the new node does not immediately follow the last selected node in guideline directed graph 206, navigation module 210 can determine that the user has skipped a node and can perform a pre-determined action. The pre-determined action may include, for example, displaying a warning, not accepting the user's node selection (e.g., not highlighting the selected node) in navigation interface 208, etc.

Besides validating the user's selection, the recorded sequences of selection of the nodes can be provided to other components of interactive medical guideline engine 202 to perform other functions to improve user experience. Specifically, interactive medical guideline engine 202 includes a patient medical history module 212 that can maintain the medical histories of different patients. Interactive medical guideline engine 202 can accept the recorded sequence of selections of a patient from navigation module 210 and store the recorded sequence as part of patient medical record 226 in database 204. Each patient medical record 226 can be associated with a patient identifier in database 204. Moreover, interactive medical guideline engine 202 also includes a data export module 214 which can export data representing the recorded sequence into an document (e.g., a patient's report, a meeting presentation, etc.) that can be part of the patient's medical record. The data may include, for example, a graphical representation of the recorded sequence.

### D. Customized Medical Guideline

Besides providing a user with access to a medical guideline, interactive medical guideline engine 202 also allows a user to create a customized version of the medical guideline. Specifically, via navigation interface 208, a user can create a customized node representing a clinical decision (e.g., a treatment, a diagnosis, etc.) not represented by any of the nodes of directed graph 206, and insert the customized node into directed graph 206 to create a customized directed graph. The directed graph can be customized for a particular patient.

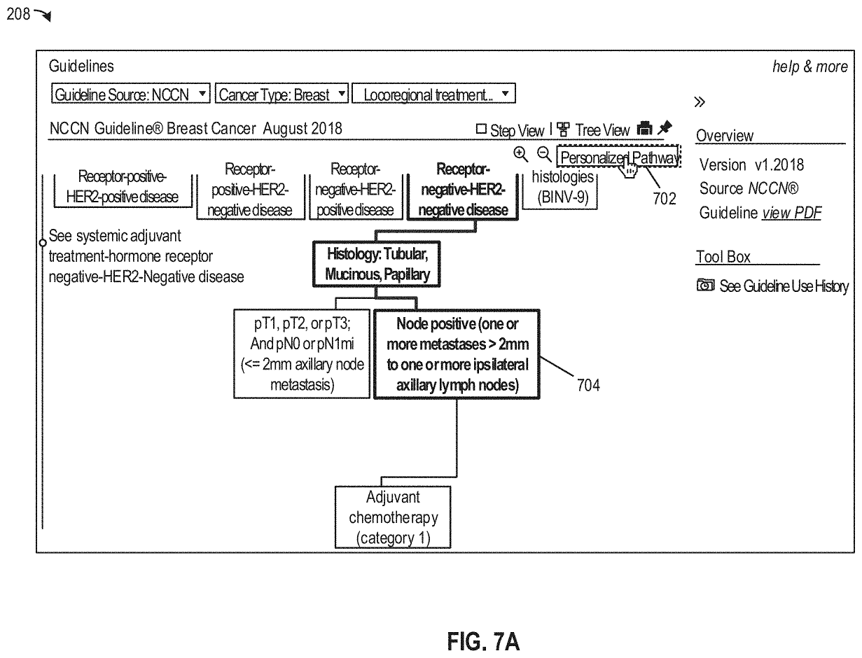

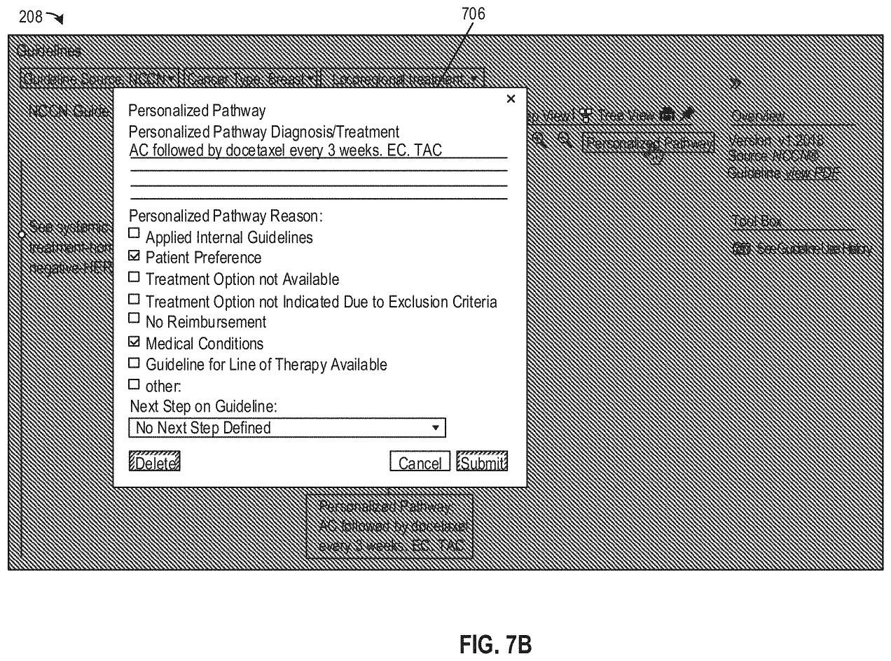

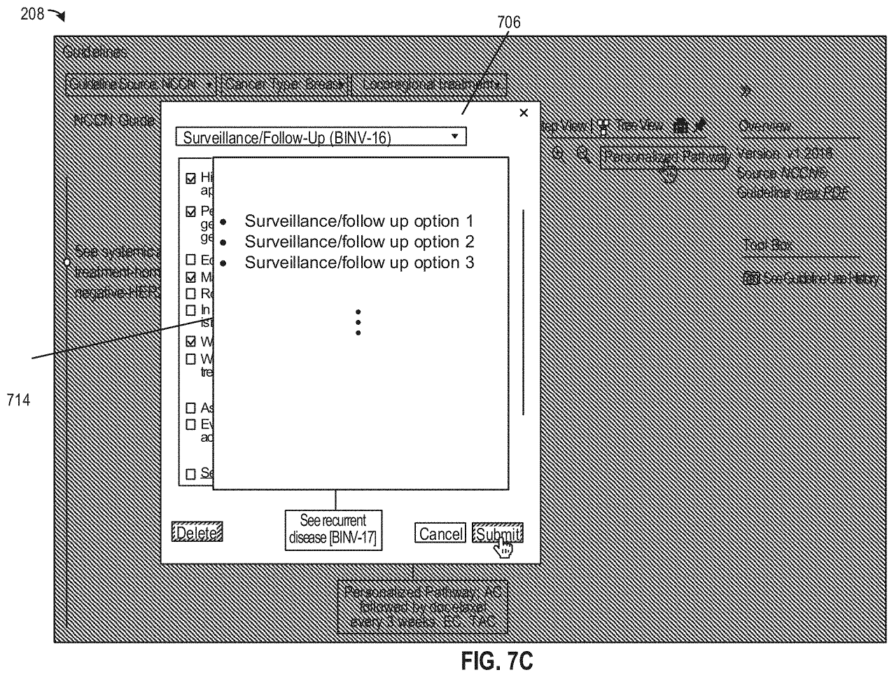

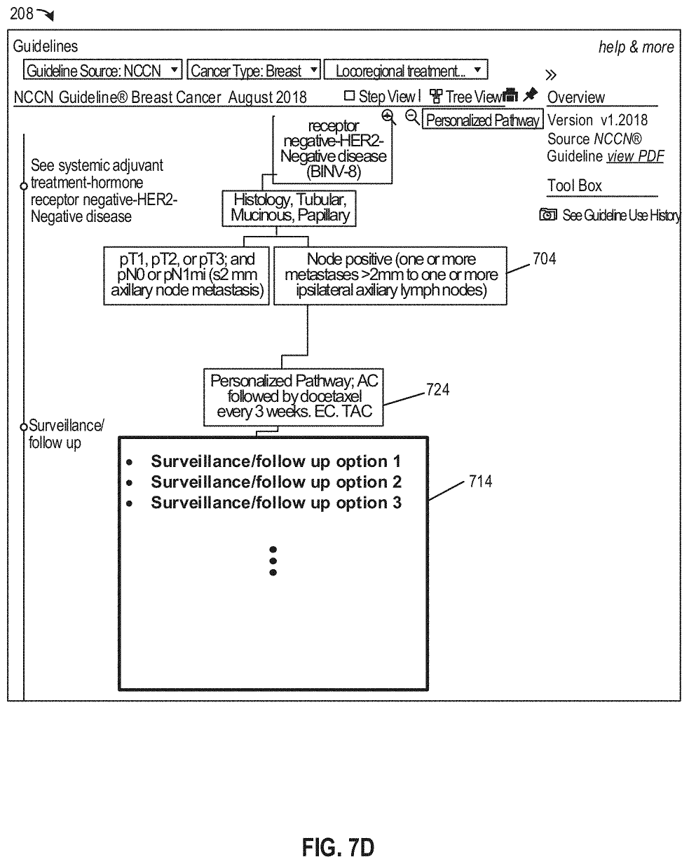

FIG. 3C illustrates an example of a customized medical guideline directed graph 206. As shown in FIG. 3C, a customized node 322 representing a personalized treatment (a clinical decision) is created and inserted between node 304 (representing diagnostic tests) and node 306 (first treatment step of lumpectomy). As part of the insertion operation, the edge 314 between node 304 and node 306 is disabled, whereas a new edge 334 is added from node 304 to node 322 and a new edge 336 is added from node 322 to node 306. Customized node 322 can be created and inserted into directed graph 206 when the user selects node 304. The user can then select customized node 322 as the next step in the patient's medical journey. The sequence of selections by the user, including the customized node 322, can be stored in patient medical record 226. As to be described below, FIG. 7A, FIG. 7B, FIG. 7C, and FIG. 7D illustrate example operations of customizing a medical guideline and to displaying the customized medical guideline via navigation interface 208.

### E. Medical Guideline Update

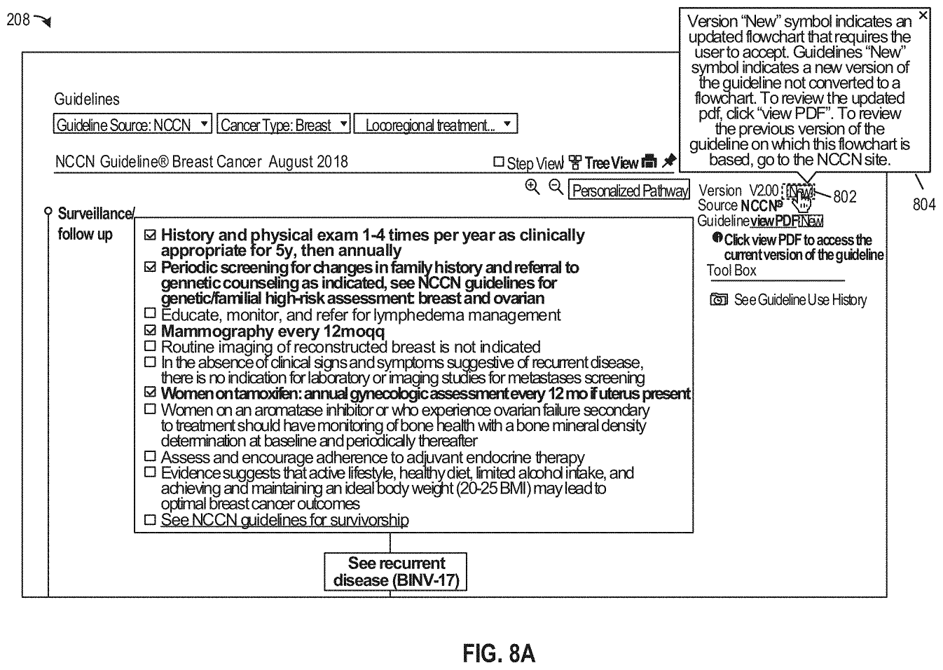

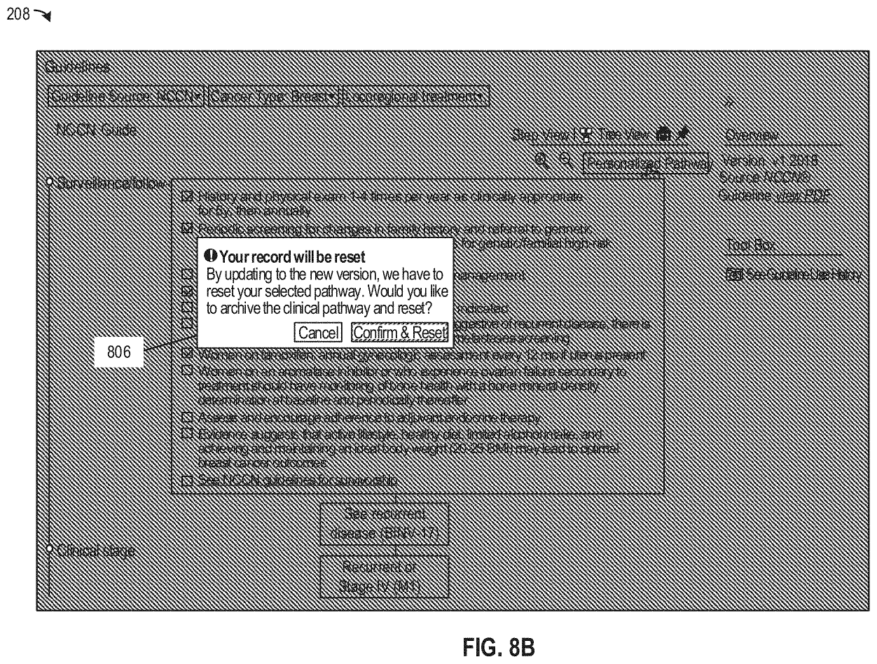

In addition, interactive medical guideline engine 202 further includes a reset module 216 that can prompt a user to remove a sequence of node selection tracked by navigation module 210. Specifically, reset module 216 can detect an update to the directed graph 206. Reset module 216 can detect the update based on, for example, detecting a change in the version number associated with directed graph 206 in database 204. Based on the detection, reset module 216 can cause navigation interface 208 to provide notifications via navigation interface 208. The notifications can indicate, for example, the version of directed graph 206 has been updated. The notifications can also prompt the user to remove the sequence of node selection for a patient based on the old medical guideline. The notifications can also prompt to user to navigate through the updated medical guideline to create a new sequence of node selection for the patient, to ensure that the clinical decisions made for the patient are based on the most up-to-date medical guideline. As to be described below, FIG. 8A and FIG. 8B illustrate example reset operations of navigation interface 208.

### III. Example Operations of Navigation Interface

A. Start Page

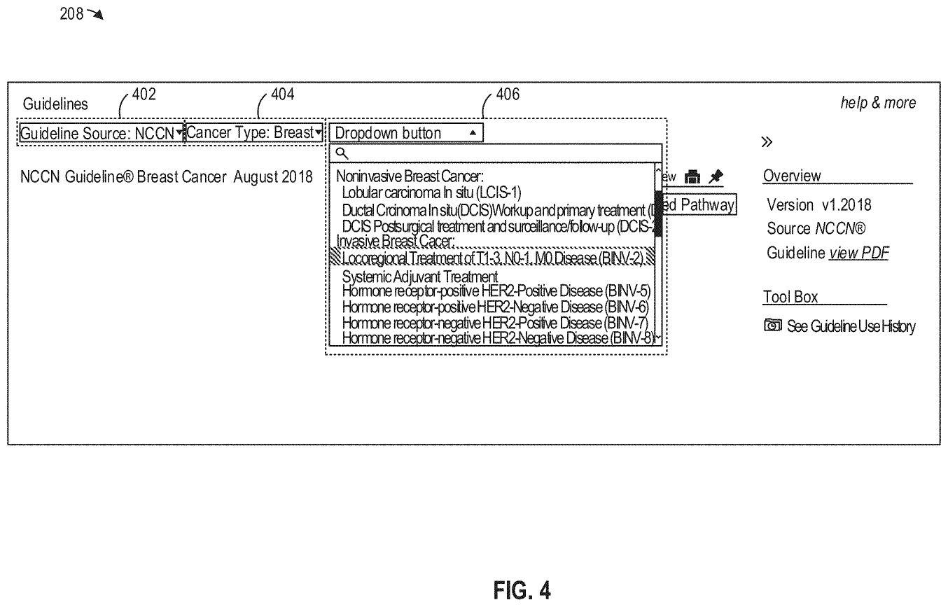

FIG. 4-FIG. 9 illustrate example operations of navigation interface 208. As shown in FIG. 4, navigation interface 208 includes drop down menus 402, 404, and 406. Drop down menu 402 allows a user to select the source of the medical guideline to be accessed, whereas drop down menu 404 allows a user to select the disease of the medical guideline to be accessed. Based on the inputs from drop down menus 402 and 404, navigation module 210 can retrieve a directed graph 206 associated with the selected source and disease in database 204, and provide the retrieved directed graph 206 for navigation in navigation interface 208. In addition, drop down menu 406 allows a user to select a node to start the navigation of the directed graph. Each selectable item in drop down menu 406 can represent a treatment step or a diagnosis step resented by a node in the directed graph.

B. Tree View

FIG. 5A, FIG. 5B, and FIG. 5C illustrate example operations of navigation interface 208 under the tree mode. As shown in FIG. 5A and FIG. 5B, navigation interface 208 includes an icon 502 labelled “step view,” an icon 504 labelled “tree view,” and an export icon 506. FIG. 5A, FIG. 5B, and FIG. 5C illustrate a tree mode operation when the user selects icon 504. As shown in FIG. 5A-FIG. 5C, under the tree mode, navigation interface 208 can display a directed graph in a visual tree 510, which includes graphical elements representing the nodes and edges of the directed graph. For example, in visual tree 510, a box can represent a node, whereas lines that connect the nodes represent edges. Each node displayed in navigation interface 208 can include the text of, for example, a treatment step, a diagnostic step, a treatment result, a diagnosis result, etc., extracted from the medical guideline document.

Some of the nodes in FIG. 5A, such as nodes 512a, 512b, 512c, 512d, 512e, and 512f, can represent a precondition for a clinical decision (e.g., a surgical level in nodes 512a and 512b, a clinical stage in node 512c, a biopsy analysis result in each of nodes 512d, 512e, and 512f). The rest of the nodes in FIG. 5A, including nodes 514a, 514b, 514c, 514d, 514e, and 514f, represent clinical decisions corresponding to the preconditions represented by nodes 512d, 512e, 512f, 512b, and 512c. The nodes are connected by edges, such as edge 516, to represent the dependency relationship (e.g., a sequential relationship, a logical relationship, etc.) between a precondition and a clinical decision.

The graphical element representing each node in the decision tree displayed in navigation interface 208 is made selectable by interactive medical guideline engine 202. The selection can reflect, for example, a treatment/diagnostic test step prescribed to and/or undertaken by a patient, a diagnosis result of the patient, etc. Navigation module 210 can detect an input indicating a selection of a node from navigation interface 208 and can update the display of the decision tree based on the selection.

In addition, as shown in FIG. 5B, navigation module 210 can cause navigation interface 208 to visually emphasize (e.g., based on different colors, tones, etc.) the selected nodes 512a, 512e, and 514b, and edges 528 and 530 that connect the selected nodes in navigation interface 208 relative to the nodes and edges that are not selected, such as node 512b and edge 534. The user's sequence of selection of nodes can be recorded in a linked list (e.g., as shown in FIG. 3A and FIG. 3B). The data of the linked list can be exported to a document (e.g., a patient's report, a meeting presentation, etc.) that can be part of the patient's medical record by data export module 214 upon selection of export icon 506. For example, data export module 214 can traverse through the linked list to obtain a precondition/clinical decision and a timestamp stored in each element, and generate a document (e.g., a presentation) that maps the precondition/clinical decisions selected for a patient, and the dates of the selection. The document can provide a timeline view of all the preconditions and clinical decisions that a user (e.g., a clinician) has selected for a patient, and can reflect the medical treatment history of the patient.

In some examples, navigation module 210 can also remove some of the unselected nodes from display in navigation interface 208 based on the user's sequence of selection of nodes, to further emphasize the preconditions and clinical decisions selected by the user and to assist the user in selecting the next step in the decision tree. For example, as shown in FIG. 5B, navigation module 210 can identify node 514b as the most recently selected node, as well as child nodes of node 514b, which include directed child nodes 536 and 538. Navigation module 210 can control navigation interface 208 to display, in addition the selected nodes 512a, 512e, and 514b, the unselected nodes 536 and 538, to provide guidance to the user of the options for the next step in the decision tree after node 514b. In addition, navigation module 210 can also control navigation interface 208 not to display nodes representing clinical decisions (based on the flags of the nodes) that are not selected in favor of the most recently selected node 514a, including nodes 514a, 514c, 514d, 514e, and 514f, to further highlight the selected clinical decision(s). But navigation module 210 can control navigation interface 208 to display nodes representing preconditions (based on the flags of the nodes) that are not satisfied, including nodes 512b, 512c, 512d, and 512f to, for example, enable the user to revisit the ranges of preconditions and to verify the basis of the clinical decisions.

Navigation interface 208 also allows a user to select a particular portion of visual tree 510 for displaying. For example, navigation interface 208 includes icons 560 which allows the user to zoom into or zoom out of visual tree 510. Navigation interface 208 can also detect a drag action of the user and move to another part of visual tree 510. Navigation interface 208 can detect a drag action based on detecting, for example, a movement of a cursor in combination of a certain key (e.g., from the keyboard) and/or a button (from a peripheral device such as a computer mouse) being pressed down. The direction of movement of the cursor can indicate, for example, a new region of a display frame (e.g., within a frame buffer) relative to the region of the display frame currently displayed in navigation interface 208. As discussed above, navigation module 210 can maintain a mapping between each node of the decision tree, as well as the connected edges, and different regions of the display frame. Based on the mapping and the newly-selected region of the display frame, navigation module 210 can identify the nodes and edges in the newly-selected region, and provide the nodes and edges for display in navigation interface 208.

FIG. 5C shows that a different set of nodes and edges from those of FIG. 5B are displayed in navigation interface 208 after detecting a drag action from the user. Based on detecting a drag action that moves the cursor up (indicated by direction A), navigation module 210 can shift down (indicated by direction B) within the display frame to identify the new region to be displayed, and identify additional nodes and edges mapped to the new region. The additional nodes include, for example, nodes 540, 542, 544, 546, and 548, as well as additional edges such as edges 550, 552, 554, etc., which are displayed in addition to nodes 514b, 536, and 538. Due to the shifting, nodes 512a-512c and edges 528 and 534 of FIG. 5B are not displayed. FIG. 5C also shows that nodes 536, 542, and 546 have been selected, and those nodes and edges 550, 552, and 554 are emphasized as a result of the selection.

In addition to drag action, navigation interface 208 can accept other types input, such as a zoom-in command or a zoom-out command detected from icon 560, to select a region of the display frame to be displayed by navigation interface 208.

C. Step View

FIG. 6A and FIG. 6B illustrates example operations of navigation interface 208 under the step mode when the user selects icon 502. As shown in FIG. 6A, in the step mode, navigation module 210 can cause navigation interface 208 to display, in a panel 602, the content of the most recently selected node of directed graph 206, which can include a diagnosis/treatment step 604. The selection of the node can be from, for example, pull down menu 406, from the visual tree 510 displayed in the tree mode, or from the selectable options of the next steps in panel 602 as to be described next.

Panel 602 also displays selectable options 606 (e.g., options 606a, 606b, and 606c) which represent the direct child nodes of the most recently selected node. The options may include a set of clinical decision options following the current clinical decision represented by the most recently selected node. In the example of FIG. 6A, the options may include, for example, different treatment responses, different diagnosis results, different treatment steps, different diagnosis steps, etc., that immediately follow the current treatment/diagnostic test step. In the example of FIG. 6A, option 606a, option 606b, and option 606c correspond to, respectively, node 512a, node 512b, and node 512c of FIG. 5A. Unlike the decision tree shown in the tree mode FIG. 5A-FIG. 5C, in the step mode the most recently selected node and the direct child nodes are displayed as boxes, while the edges between the nodes are not displayed.

Referring to FIG. 6B, upon receiving a selection of one of the options (e.g., option 606a), navigation module 210 can change the display of the current step as well as the possible options of the next step in navigation interface 208 based on the selected node and the location of the selected node in the directed graph. For example, in FIG. 6B, a new step 604 (corresponding to node 514b of FIG. 5B) is displayed, and new options 616a and 616b, which correspond to nodes 536 and 538 of FIG. 5B, are also displayed. In addition, navigation interface 208 also includes a panel 620 to display a sequence of the steps the user has selected, which can further assist the user in navigating through the medical guideline and in selecting the next step.

D. Customized Medical Guideline

As discussed above, interactive medical guideline engine 202 allows a user to edit directed graph 206 by adding a step not defined in the corresponding medical guideline. The step may include a precondition (e.g., a treatment response, a diagnosis result, etc.), a clinical decision (e.g., a treatment step, a diagnostic test step, etc.). FIG. 7A, FIG. 7B, FIG. 7C, and FIG. 7D illustrate example operations of customizing a medical guideline and to displaying the customized medical guideline via navigation interface 208. As shown in FIG. 7A, navigation interface 208 includes an icon 702, which can be activated to create a customized clinical decision. The customized clinical decision can be represented by a customized node, and the customized node can be added as a direct child node of the most recently selected node, which is node 704 in FIG. 7A.

As shown in FIG. 7B, upon receiving a selection of icon 702, navigation interface 208 can display a pop up window 706 that allows the user to input the content of the customized clinical decision, such as treatment details, reasons for the treatment, etc. Pop up window 706 also allows the user to select the direct child node of the customized node as the next step to be selected after the customized clinical decision. FIG. 7C shows that a node 714 from the original medical guideline, which represents a set of surveillance/follow up options, is selected as the next step. FIG. 7D illustrates that customized node 724, which represents the customized clinical decision, is added between node 704 and node 714. The user's sequence of selection of nodes from the customized directed graph, including the selection of customized node 724, can be recorded as part of the patient's medical treatment history as discussed above in FIG. 3B and FIG. 3C.

E. Medical Guideline Update

FIG. 8A and FIG. 8B illustrate example reset operations of navigation interface 208. As shown in FIG. 8A, when reset module 216 detects a change in the version number associated with directed graph 206 in database 204, reset module 216 can cause navigation interface 208 to provide a notification to indicate that the version of directed graph 206 has been updated. The notification can be in the form of a selectable icon 802 to indicate the version of directed graph 206 has been updated. When reset module 216 detects that the cursor is positioned over selectable icon 802, reset module 216 can cause navigation interface 208 to display a pop up message 804 explaining that the version of directed graph 206 has been updated and prompt the user to start using the latest version of the directed graph 206.

FIG. 8B illustrates another pop up message 806 displayed by navigation interface 208 when reset module 216 detects that the user selects (e.g., clicks on) selectable icon 802. Pop message 806 can prompt to user to erase the recorded sequence of selection of the nodes, and to navigate through the updated medical guideline directed graph 206 to create a new sequence of node selection for the patient, to ensure that the clinical decisions made for the patient are based on the most up-to-date medical guideline.

### IV. Method

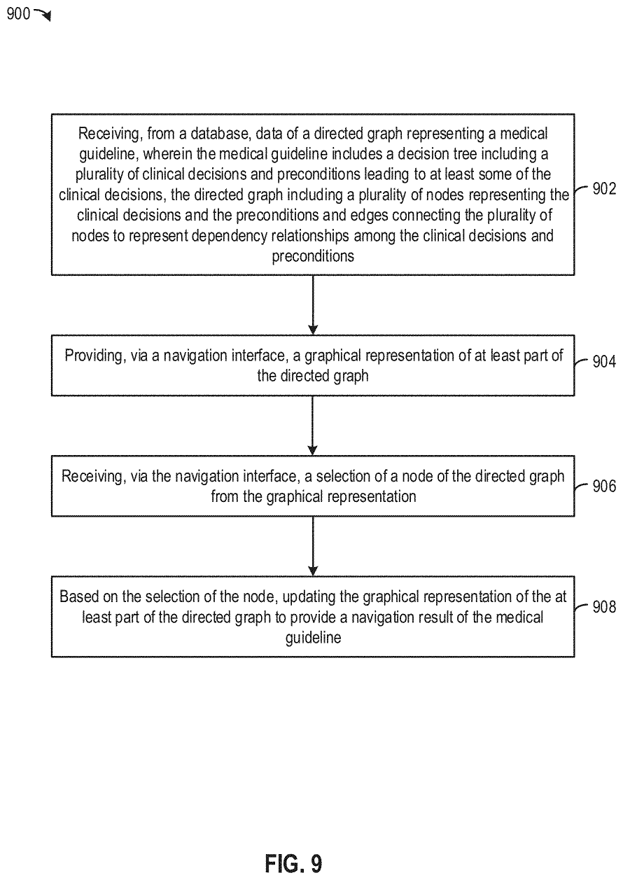

FIG. 9 illustrates a method 900 for providing an interactive medical guideline. Method 900 can be performed by, for example, interactive medical guideline engine 202.

In step 902, navigation module 210 of interactive medical guideline engine 202 receives, from a database (e.g., database 204), data of a directed graph (e.g., directed graph 206) representing a medical guideline, such as the example medical guideline illustrated in FIG. 1A and FIG. 1B. The medical guideline includes a decision tree including a plurality of clinical decisions and preconditions leading to at least some of the clinical decisions. The directed graph includes a plurality of nodes representing the clinical decisions and the preconditions and edges connecting the plurality of nodes to represent dependency relationships among the clinical decisions and preconditions.

An example of directed graph 206 is illustrated in FIG. 3A. In directed graph 206, each node can represent a treatment step, a diagnostic test step, a result of the treatment step, a diagnosis result, etc. Each node can also be associated with a flag (enclosed in square brackets) indicating whether the node is a precondition (e.g., where the node represents a result of the treatment step, a diagnosis result, etc.) or a clinical decision when the precondition is satisfied (e.g., to perform a treatment step, a diagnostic test step, etc.). Each edge can represent a dependency relationship among the nodes, which in turn can reflect, for example, a sequential relationship between two steps, a logical relationship between a precondition and a clinical decision, etc. Directed graph 206 can be generated by extracting the guideline information from a medical guideline document. The extraction can be performed manually, by an automated process, or by a combination of both.

In step 904, navigation module 210 provides, via navigation interface 208 of interactive medical guideline engine 202, a graphical representation of at least part of the directed graph.

In some examples, as shown in FIG. 5A to FIG. 5C, the graphical representation can include a visual tree including graphical elements representing at least some of the plurality of nodes. The graphical elements representing the nodes are selectable via the navigation interface and include text of the clinical decisions and the preconditions represented by the nodes. The visual tree can be displayed when navigation interface 208 operates under the tree mode.

Navigation module 210 can also maintain a mapping between each node of the decision tree and different locations within a display frame (e.g., within a frame buffer). Navigation module 210 can identify nodes mapped to a pre-determined region of the display frame, and provide the identified nodes to navigation interface 208 for graphical representation. The mapping allows a user to control navigation interface 208 to zoom into or out of a node or a set of nodes of the decision tree, to move to a different part of the decision tree that is not currently displayed in navigation interface 208, etc.

In some examples, as shown in FIG. 6A and FIG. 6B, the graphical representation can include boxes representing the most recently selected node and its direct child nodes. The direct child nodes can represent, for example, all possible options of the next clinical decision, all possible treatment responses/diagnosis results, etc. No edge between the nodes is displayed. The boxes can be displayed when navigation interface 208 operates under the step mode. The step mode allows the user to traverse the directed graph one node at a time.

In step 906, navigation module 210 receives, via navigation interface 208, a selection of a node of the directed graph from the graphical representation. When operating under a tree mode where a visual tree is displayed, navigation module 210 can detect selection of a new node from the visual tree. Navigation module 210 can also validate the selection of a new node in the tree mode based on determining whether the new node is a direct child node of the last selected node. If the new node is not the direct child node of the last selected node, navigation module 210 can perform an action, such as displaying a warning via navigation interface 208, not accepting the selection, etc. In addition, as shown in FIG. 7A-FIG. 7C, navigation module 210 can also allow a user to create a customized node representing a customized clinical decision not defined in the medical guideline, insert the customized node in the directed graph, and select the customized node. On the other hand, when operating under the step mode where boxes representing the most recently selected node and its direct child nodes are displayed, a user can select one of the boxes representing the directed child nodes as the new node, as shown in FIG. 6A and FIG. 6B. In both the tree mode and the step mode, navigation module 210 can record and track a sequence of selection of the nodes via the navigation interface, and use the sequence to, for example, validate a selection of a new node, to export to a document of a clinical decision history of a patient, etc.

In step 908, based on the selection of the node, navigation module 210 can update the graphical representation of the at least part of the directed graph to provide a navigation result of the medical guideline. For example, in the tree mode, navigation module 210 can visually emphasize (e.g., based on font size, color, line width, etc.) the node and the edge leading to the node relative to other unselected nodes and edges, as shown in FIG. 5B and FIG. 5C. Moreover, in the step mode, based on the selected node and the location of the selected node in the directed graph, navigation module 210 can change the display of the currently-selected node and the direct child nodes in navigation interface 208, as shown in FIG. 6A and FIG. 6B.

### V. Computer System

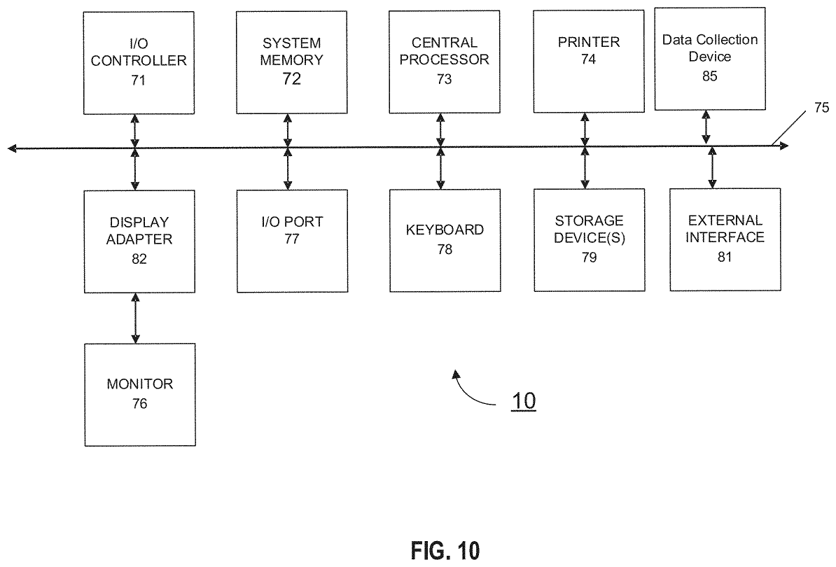

Any of the computer systems mentioned herein, such as system 200 of FIG. 2, may utilize any suitable number of subsystems. Examples of such subsystems are shown in FIG. 10 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 10 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

What is claimed is:

1. A computer-implemented method of providing an interactive medical guideline, comprising:
receiving, from a database, data of a directed graph representing a medical guideline, wherein the medical guideline includes a decision tree including a plurality of clinical decisions and preconditions leading to at least some of the clinical decisions, the directed graph including a plurality of nodes representing the clinical decisions and the preconditions and edges connecting the plurality of nodes to represent dependency relationships among the clinical decisions and the preconditions;
providing, via a navigation interface, a graphical representation of at least part of the directed graph;
receiving, via the navigation interface, a selection of a node of the directed graph from the graphical representation; and
based on the selection of the node, updating the graphical representation of the at least part of the directed graph to provide a navigation result of the medical guideline.

2. The method of claim 1, wherein the graphical representation comprises a visual tree including graphical elements representing at least some of the plurality of nodes, wherein the graphical elements representing the nodes are selectable via the navigation interface and include text of the clinical decisions and the preconditions represented by the nodes.

3. The method of claim 2, further comprising:
responsive to receiving the selection, highlighting the node and an edge leading to the node, to indicate a sequence of clinical decisions made for a patient.

4. The method of claim 2, further comprising:
mapping the plurality of nodes of the decision tree to different regions of a display frame;
receiving, via the navigation interface, an input to select a region of the display frame to be displayed in the navigation interface; and
based on the selected region and the mapping, providing the graphical representation of at least part of the directed graph including the nodes mapped to the selected region via the navigation interface.

5. The method of claim 4, wherein the input comprises at least one selected from: a zoom-in command, a zoom-out command, or a drag action.

6. The method of claim 1, wherein the graphical representation comprises only a currently-selected node of the directed graph and all direct child nodes of the currently-selected node.

7. The method of claim 6, wherein each of the currently-selected node and the direct child nodes is represented as a box including text of the clinical decisions and the preconditions represented by the currently-selected node and the direct child nodes in the graphical representation.

8. The method of claim 7, further comprising:
receiving a selection to select between a tree view or a step view,
wherein the graphical representation comprising only the currently-selected node of the directed graph and all the direct child nodes of the currently-selected node is provided based on the selection of the step view.

9. The method of claim 1, further comprising:
recording a sequence of selection of the nodes from the graphical representation and timestamps indicating dates of selection of the nodes in a linear data structure.

10. The method of claim 9, further comprising:
traversing the linear data structure to obtain the sequence of selection and the timestamps; and
exporting the sequence of selection of the nodes and the timestamps to a document of a history of clinical decisions of a patient.

11. The method of claim 9, further comprising:
receiving a selection of a new node;
determining a last selected node from the sequence;
validating the selection of the new node based on determining whether the new node is a direct child node of the last selected node in the directed graph; and
performing an action based on a result of the validation.

12. The method of claim 9, further comprising:
detecting that a version of the directed graph has been updated; and
based on the detection, providing a notification via the navigation interface to erase the recorded sequence.

13. The method of claim 1, further comprising:
receiving, via the navigation interface, a request to insert a node representing a customized clinical decision not defined in the medical guideline into the directed graph;
responsive to the request, generating a pop up window to collect information of the customized clinical decision;
generating a customized node including the collected information; and
inserting, based on the collected information, the customized node into the directed graph to generate a customized directed graph.

14. The method of claim 13, wherein the information includes the customized clinical decision to be represented by the customized node, and a node of the directed graph which is to become a direct child node of the customized node.

15. A computer product comprising a computer readable medium storing a plurality of instructions for controlling a computer system to perform a method of providing an interactive medical guideline, the method comprising:
receiving, from a database, data of a directed graph representing a medical guideline, wherein the medical guideline includes a decision tree including a plurality of clinical decisions and preconditions leading to at least some of the clinical decisions, the directed graph including a plurality of nodes representing the clinical decisions and the preconditions and edges connecting the plurality of nodes to represent dependency relationships among the clinical decisions and the preconditions;
providing, via a navigation interface, a graphical representation of at least part of the directed graph;
receiving, via the navigation interface, a selection of a node of the directed graph from the graphical representation; and
based on the selection of the node, updating the graphical representation of the at least part of the directed graph to provide a navigation result of the medical guideline.

16. The computer product of claim 15, wherein the graphical representation comprises a visual tree including graphical elements representing at least some of the plurality of nodes, wherein the graphical elements representing the nodes are selectable via the navigation interface and include text of the clinical decisions and the preconditions represented by the nodes.

17. The computer product of claim 16, wherein the method further comprises:
mapping the plurality of nodes of the decision tree to different regions of a display frame;
receiving, via the navigation interface, an input to select a region of the display frame to be displayed in the navigation interface; and
based on the selected region and the mapping, providing the graphical representation of at least part of the directed graph including the nodes mapped to the selected region via the navigation interface.

18. The computer product of claim 15, wherein the graphical representation comprises only a currently-selected node of the directed graph and all direct child nodes of the currently-selected node.

19. The computer product of claim 15, wherein the method further comprises:
recording a sequence of selection of the nodes from the graphical representation and timestamps indicating dates of selection of the nodes in a linear data structure.

20. The computer product of claim 15, wherein the method further comprises:
receiving, via the navigation interface, a request to insert a node representing a customized clinical decision not defined in the medical guideline into the directed graph;
responsive to the request, generating a pop up window to collect information of the customized clinical decision;
generating a customized node including the collected information; and
inserting, based on the collected information, the customized node into the directed graph to generate a customized directed graph.
